(19) United States
(12) Patent Application Publication
(10) Pub. No.: US 2026/0315634 A1
(43) Pub. Date: Oct. 15, 2026

(54) APPARATUS AND METHOD FOR FLUSHING, CLEANING, AND DECONTAMINATING GRAFTS AND STENTS

(71) Applicants: Kimberley Levy, Fort Myers, FL (US); Frank Levy, Fort Myers, FL (US)

(72) Inventors: Kimberley Levy, Fort Myers, FL (US); Frank Levy, Fort Myers, FL (US)

(21) Appl. No.: 19/647,150

(22) Filed: Apr. 14, 2026

Related U.S. Application Data
(60) Provisional application No. 63/788,507, filed on Apr. 14, 2025.

(51) Int. Cl. A61L 2/20; A61L 103/15

(52) U.S. Cl. CPC A61L 2/20; A61L 2103/15; A61L 2202/15

(57) ABSTRACT

A flushing, cleaning, and decontaminating apparatus for flushing, cleaning, and decontaminating grafts and stents includes a multi-channel arrangement at a proximal first end thereof and a Venturi-agitating tip assembly at a tip at a distal second end thereof. A compressed supply unit is fluidly connected to the multi-channel arrangement for supplying pressurized medical carbon dioxide (CO₂) to the Venturi-agitating tip assembly. An additional flushing, cleaning, and decontaminating solution is fluidly connected to the multi-channel arrangement for supplying additional flushing, cleaning, and decontaminating solution to the Venturi-agitating tip assembly. The pressurized medical carbon dioxide (CO₂) from the compressed supply unit and the additional flushing, cleaning, and decontaminating solution are combined within the Venturi-agitating tip assembly in a manner generating a cleaning medium that is ultimately dispensed from therefrom.

### CROSS REFERENCE TO RELATED APPLICATION

This application claims the benefit of U.S. Provisional Patent Application 63/788,507, entitled “APPARATUS AND METHOD FOR FLUSHING, CLEANING, AND DECONTAMINATING GRAFTS AND STENTS,” filed Apr. 14, 2025, which is incorporated herein by reference.

### BACKGROUND

### 1. Field of the Invention

This invention relates to an apparatus and process for flushing, cleaning, and decontaminating grafts and stents.

### 2. Description of the Related Art

It has been found that rinsing of grafts and stents prior to implantation results in better outcomes. The present invention provides an apparatus and process for flushing, cleaning, and decontaminating grafts and stents.

### SUMMARY OF THE INVENTION

In one aspect a flushing, cleaning, and decontaminating apparatus for flushing, cleaning, and decontaminating grafts and stents is disclosed. The apparatus comprises a multi-channel arrangement at a proximal first end thereof and a Venturi-agitating tip assembly at a tip at a distal second end thereof. A compressed supply unit is fluidly connected to the multi-channel arrangement for supplying pressurized medical carbon dioxide (CO₂) to the Venturi-agitating tip assembly. An additional flushing, cleaning, and decontaminating solution is fluidly connected to the multi-channel arrangement for supplying additional flushing, cleaning, and decontaminating solution to the Venturi-agitating tip assembly. The pressurized medical carbon dioxide (CO₂) from the compressed supply unit and the additional flushing, cleaning, and decontaminating solution are combined within the Venturi-agitating tip assembly in a manner generating a cleaning medium that is ultimately dispensed from therefrom.

In some embodiments the flushing, cleaning, and decontaminating apparatus allows each of the pressurized CO₂ and additional flushing, cleaning, and decontaminating solution to be used individually.

In some embodiments the compressed supply unit includes an inlet port to which a pressurized gas cylinder containing medical carbon dioxide (CO₂) is selectively connected, and an outlet port in communication with the inlet port, and ultimately the pressurized gas cylinder containing medical carbon dioxide (CO₂).

In some embodiments the outlet port is provided with a Luer connection for secure and selective attachment of an outlet tube, and the outlet tube is secured to the multi-channel arrangement for the supply of pressurized medical carbon dioxide (CO₂) to thereto.

In some embodiments outlets at a tip of the Venturi-agitating tip assembly are oriented such that a flushing, cleaning, and decontaminating suspension is sprayed at an angle of 25 degrees to 65 degrees relative to a longitudinal axis of the Venturi-agitating tip assembly to ensure the flushing, cleaning, and decontaminating suspension is sprayed directly onto an inner wall (or exterior) of the graft or stent being treated and is not entrained within fluid flowing.

In some embodiments the multi-channel arrangement includes a dual lumen catheter that connects the Venturi-agitating tip assembly to the pressurized medical carbon dioxide (CO2) from the compressed supply unit and the additional flushing, cleaning, and decontaminating solution from a syringe.

In some embodiments the syringe includes a one-way valve at its outlet to ensure that additional flushing, cleaning, and decontaminating solution from the syringe only flows out of the syringe.

In some embodiments a first stopcock is positioned between a micro-hose and a first lumen of the dual lumen catheter.

In some embodiments the first stopcock allows for three flow options comprising a first flow option for free flow between the micro-hose and the first lumen of the dual lumen catheter, a second option for the flow from the micro-hose to a discharge tube that is used during purging procedures, and a third option blocking the flow at the end of the micro-hose.

In some embodiments the additional flushing, cleaning, and decontaminating solution is delivered to a second lumen of the dual lumen catheter at the proximal first end thereof, and ultimately to the Venturi-agitating tip assembly, via a container, in particular, the syringe, connected to the second lumen of the dual lumen catheter by a supply line.

In some embodiments a second stopcock is positioned between the syringe and the second lumen of the dual lumen catheter.

In some embodiments the second stopcock allows for three flow options comprising a first flow option for free flow between the syringe and the second lumen of the dual lumen catheter, a second option for the flow from the syringe to a discharge tube that is used during purging procedures, and a third option blocking the flow at the end of the syringe.

In some embodiments the additional flushing, cleaning, and decontaminating solution is delivered to a second lumen of the dual lumen catheter at the proximal first end thereof, and ultimately to the Venturi-agitating tip assembly, via a container, in particular, the syringe, connected to the second lumen of the dual lumen catheter by a supply line.

In some embodiments the proximal first end of the Venturi-agitating tip assembly includes a first and second inputs for attachment to a dual lumen catheter.

In some embodiments the first and second inputs respectively lead to a first channel and a second channel of the Venturi-agitating tip assembly, and the first and second channels lead to, and are in fluid communication with, a mixing chamber located in a central portion of the Venturi-agitating tip assembly, that is, between the proximal first end and the distal second end.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view showing the flushing, cleaning, and decontaminating apparatus for flushing, cleaning, and decontaminating grafts and stents showing a graft being treated.

FIG. 1A is a perspective view showing the flushing, cleaning, and decontaminating apparatus for flushing, cleaning, and decontaminating grafts and stents showing a stent being treated.

FIG. 2 is a schematic of the flushing, cleaning, and decontaminating apparatus.

FIG. 3 is a schematic showing the internal lumens of the dual lumen catheter as they connect to the Venturi-agitating tip assembly.

FIGS. 4A, 4B, and 4C respectively show a longitudinal cross sectional view, a perspective view, and a perspective cross sectional view of a Venturi-agitating tip assembly in accordance with a first embodiment.

FIGS. 5A, 5B, 5C, and 5D respectively show a perspective view, a longitudinal cross-sectional perspective view, an exploded view, and a lateral cross-sectional view of a Venturi-agitating tip assembly in accordance with a second embodiment.

FIGS. 6A, 6B, 6C, 6D, and 6E respectively show a perspective view, an exploded view, a front partial cross-sectional view, a rear partial cross-sectional view, and a lateral cross-sectional view of a Venturi-agitating tip assembly in accordance with a third embodiment.

FIGS. 7A, 7B, 7C, 7D, 7E, and 7F respectively show a side view, a top view, a detailed side view, a perspective view, a longitudinal cross sectional view, and a lateral cross sectional view showing an embodiment of a Venturi-agitating tip assembly in accordance with a fourth embodiment.

### DETAILED DESCRIPTION

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

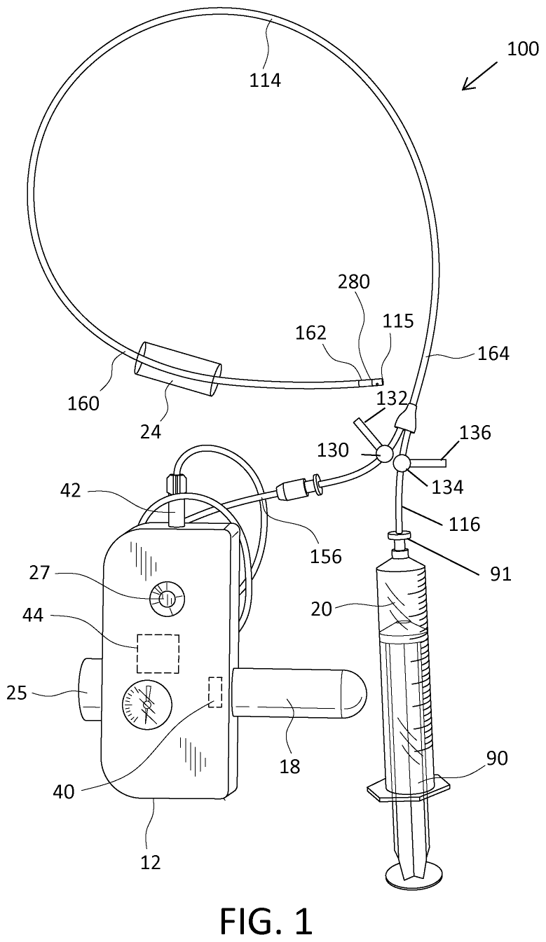

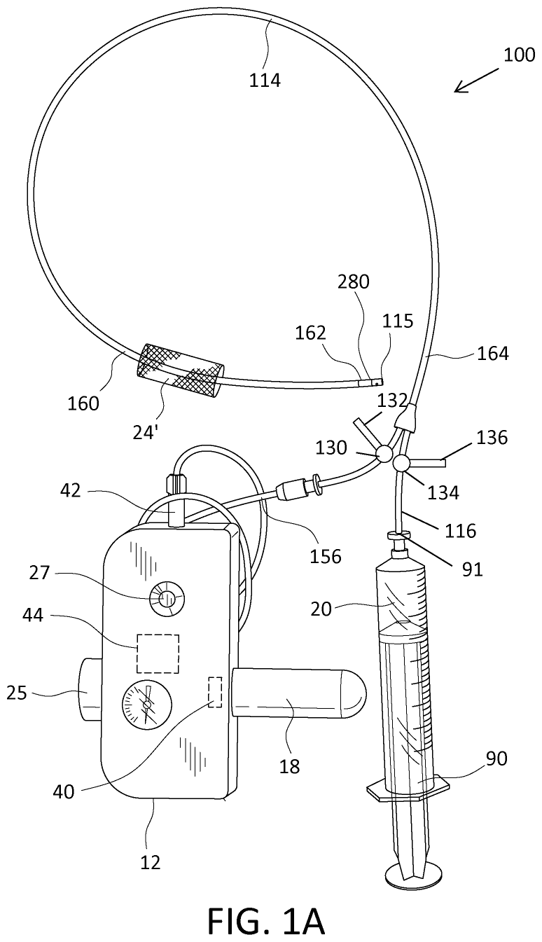

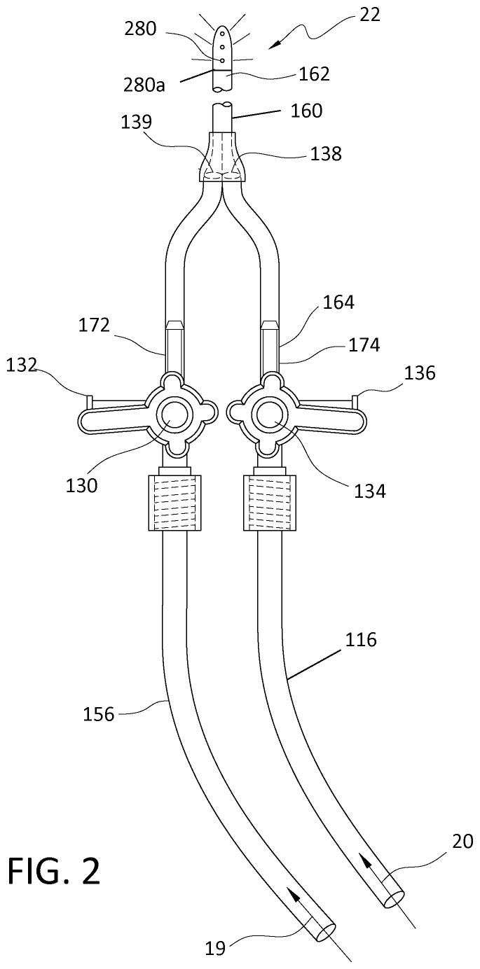

Referring to FIGS. 1, 2, and 3, the flushing, cleaning, and decontaminating apparatus 100 for flushing, cleaning, and decontaminating grafts and stents 24, 24′ is composed of a multi-channel arrangement 114 at a proximal first end thereof and a tip 115 at a distal second end thereof. While the apparatus is disclosed for use with grafts and stents, it is appreciated that the apparatus may be used with other small, cylindrical implants well suited for the apparatus's ability to conveniently and reliably spray such implants.

The apparatus 100 dispenses individual amounts of medical grade CO₂, saline, or other solutions, as well as combinations thereof, in a sterile manner, to grafts and stents before they are placed in the body. Use of the present apparatus helps to avoid contaminants and bacteria that are introduced into the body, wherein such contaminants cause many complications (e.g., 20% to 30% of cerebral strokes, air embolism, thrombosis, etc.). The apparatus is also safe, portable, and easy and quick to set up, as it replaces large unsanitized, contaminated, and bulky tanks that sometimes require piped in hosing through the walls of an additional room from the operating room and may cause mistakes with delivering the wrong gases.

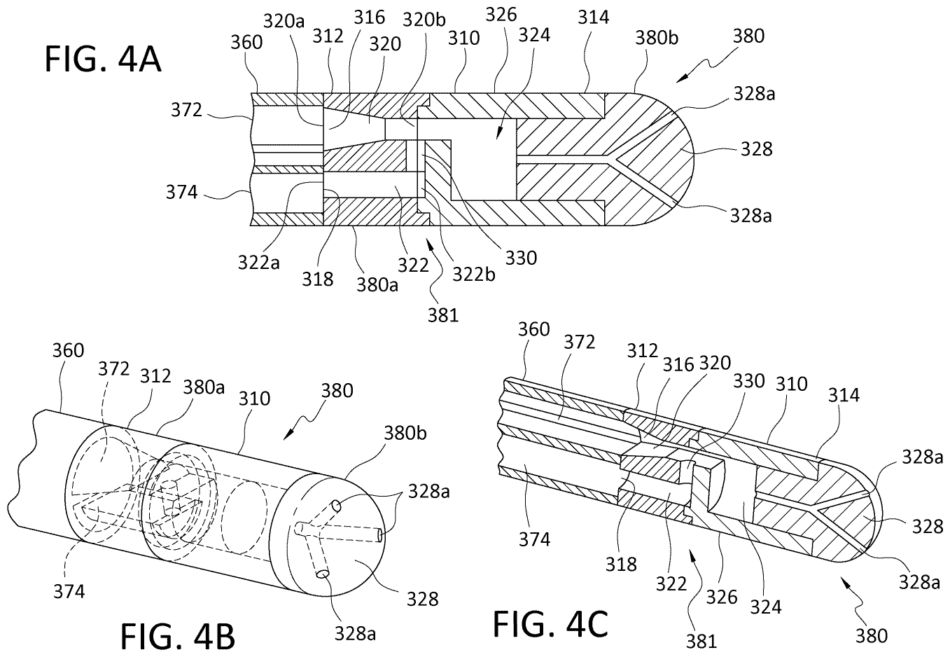

The flushing, cleaning, and decontaminating apparatus 100 includes a Venturi-agitating tip assembly 280 at the tip 115 thereof and the multi-channel arrangement 114 is coupled to the proximal first end of the Venturi-agitating tip assembly 280. The flushing, cleaning, and decontaminating apparatus 100 also includes a compressed supply unit 12 fluidly connected to the multi-channel arrangement 114 for supplying pressurized medical carbon dioxide (CO₂) 19 to the proximal first end 280a of a Venturi-agitating tip assembly 280 and an additional flushing, cleaning, and decontaminating solution 20 fluidly connected to the multi-channel arrangement 114 for supplying additional flushing, cleaning, and decontaminating solution 20 to the proximal first end 280a of the Venturi-agitating tip assembly 280. FIGS. 1, 2, and 3 generally disclose the Venturi-agitating tip assembly, while FIGS. 4A-C, 5A-D, 6A-E, and 7A-F disclose various embodiments of the Venturi-agitating tip assembly in detail.

The compressed supply unit 12 supplies pressurized medical carbon dioxide (CO₂) and is fluidly connected to the multi-channel arrangement 114. The compressed supply unit 12 receives gas at a very high pressure and reduces the pressure of the gas in a controlled manner for use in accordance with the present flushing, cleaning, and decontaminating apparatus 100. Pressurized medical carbon dioxide (CO₂), from the compressed supply unit 12, and the additional flushing, cleaning, and decontaminating solution 20 are combined within the Venturi-agitating tip assembly 280 in a manner generating a cleaning medium that is ultimately dispensed from therefrom. A method in accordance with the apparatus is also disclosed. As discussed below, in addition to the combination of pressurized CO₂ 19 and additional flushing, cleaning, and decontaminating solution 20 being utilized, the present system allows each of the pressurized CO₂ 19 and additional flushing, cleaning, and decontaminating solution 20 to be used individually.

With reference to FIG. 1, the compressed supply unit 12 is disclosed. The compressed supply unit 12 includes a supply unit as disclosed in detail in U.S. Pat. No. 9,427,522, filed Sep. 26, 2014 and issued Aug. 30, 2016, entitled “DELIVERY SYSTEM FOR THE EFFECTIVE, RELIABLE AND FOOLPROOF DELIVERY OF CONTROLLED AMOUNTS OF A MEDICAL FLUID,” which is incorporated herein by reference. The compressed supply unit 12 delivers pressurized gas that has been regulated to a desired pressure, in particular medical carbon dioxide (CO₂), which then forces the pressurized carbon dioxide (CO₂) into a delivery catheter. With reference to FIG. 1, the compressed supply unit 12 includes an inlet port 40 to which a source of pressurized medical carbon dioxide (CO₂), for example, a pressurized gas cylinder 18 containing medical carbon dioxide (CO₂) 19 is selectively connected, and an outlet port 42 in communication with the inlet port 40, and ultimately the at least one pressurized gas (CO₂) cylinder 18. While disclosed embodiment employs a specific supply unit to control the pressure of the CO₂ being used in accordance herewith, it is appreciated that various mechanisms for delivering CO₂ at an optimal pressure may be used.

The pressurized gas cylinder 18 is secured to, and maintained in fluid communication with, the integrated compressed supply unit 12 by a cylinder cartridge puncture valve and a fitting formed at the inlet port 40 of the compressed supply unit 12. In accordance with a preferred embodiment, the cylinder cartridge puncture valve has a mechanism for piercing the pressurized gas cylinder 18, as is known in the art, and for holding or securing the pressurized gas cylinder 18 in place.

The pressurized gas, that is, pressurized medical carbon dioxide (CO₂) 19, exits the inlet port 40 and passes through a regulator valve assembly 44 controlled by a press button actuator 27 and regulator adjustment dial 25. The regulator valve assembly 44 dictates the pressure of the gas as it ultimately exits the outlet port 42. In accordance with a preferred embodiment, the regulator valve assembly 44 has a selective outlet pressure in the range of 7 psi (pounds per square inch) to 19 psi, which, relative to the pressure coming from a traditional large gas tank (commonly 700-900 psi), is a relatively low pressure. The desired outlet pressure is achieved by rotating the regulator adjustment dial 25 associated with the press button actuator 27 until the desired pressure is achieved as identified via the PSI pressure gauge. In addition to regulating the applied pressure, the regulator valve assembly 44 also includes a valve (not shown) that controls the passage of gas from the inlet port 40 to the outlet port 42. The valve is controlled via a press button actuator 27 such that a user may selectively determine when gas may pass therethrough to the outlet port 42.

As mentioned above, the regulator valve assembly 44 also includes a regulator adjustment dial 25, which controls the pressure of the medical carbon dioxide (CO₂) permitted to exit the outlet port 42 by either rotating the regulator adjustment dial 25 clockwise or counterclockwise as may be desired to adjust the applied pressure. The applied pressure is monitored using the PSI pressure gauge formed on the front face of the compressed supply unit 12.

In practice, a pressurized gas cylinder 18 is supplied by the compressed supply unit 12 in the following manner. The regulator adjustment dial 25 is first rotated to its off position, at which point the compressed supply unit 12 is in its fully closed orientation. The pressurized gas cylinder 18 is then screwed into the fitting and the cylinder cartridge puncture valve punctures the pressurized gas cylinder 18. The regulator adjustment dial 25 is then turned clockwise to open the compressed supply unit 12 and allow for flow therethrough. The compressed supply unit 12 is then actuated by twisting the regulator adjustment dial 25 in a clockwise direction until a desired pressure is established. The compressed supply unit 12 is then operated as described above, through the manipulation of the press button actuator 27 and the regulator adjustment dial 25.

As mentioned above, the outlet port 42 is in fluid communication with the inlet port 40 for transport of pressurized medical carbon dioxide (CO₂) 19 in accordance with the present invention. The outlet port 42 is provided with a Luer connection for the secure and selective attachment of an outlet tube (for example, a micro-hose 156 as discussed below) thereto. The outlet tube 156 is secured to the multi-channel arrangement 114 for the supply of pressurized medical carbon dioxide (CO₂) 19 thereto.

As briefly mentioned above, the pressurized gas cylinder 18 is secured to the compressed supply unit 12 by a cartridge puncture valve as is commonly known. Pressurized medical carbon dioxide (CO₂) 19 leaves the regulator valve assembly 44 at the regulator adjusted pressure and goes to the outlet port 42.

In accordance with an alternative embodiment, an integrated compressed supply unit 12 as disclosed in U.S. Pat. No. 9,662,435, filed Sep. 26, 2014 and issued May 30, 2017, entitled “SYSTEM AND METHOD FOR THE EFFECTIVE, RELIABLE AND FOOLPROOF DELIVERY OF CONTROLLED AMOUNTS OF A MEDICAL FLUID,” which is incorporated herein by reference, may be utilized in accordance with the present invention instead of the supply unit described above.

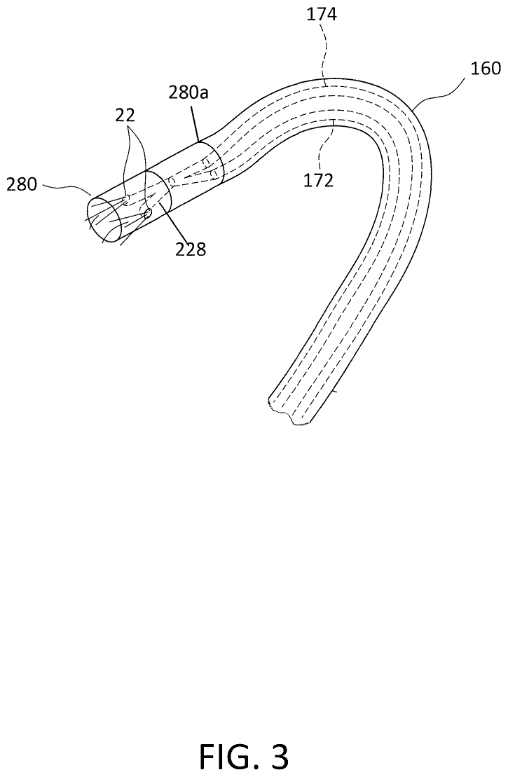

Pressurized medical carbon dioxide (CO₂) 19, from the compressed supply unit 12, and the additional flushing, cleaning, and decontaminating solution 20 are combined within the Venturi-agitating tip assembly 280 in a manner generating a flushing, cleaning, and decontaminating suspension 22 that is ultimately dispensed from the flushing, cleaning, and decontaminating apparatus 10. The flushing, cleaning, and decontaminating suspension 22 is then sprayed from the Venturi-agitating tip assembly 280. In accordance with a preferred embodiment, and with reference to FIG. 3, outlets 228 at the tip of the Venturi-agitating tip assembly 280 are oriented such that the flushing, cleaning, and decontaminating suspension 22 is sprayed at an angle of 25 degrees to 65 degrees, preferably 45 degrees, relative to the longitudinal axis of the Venturi-agitating tip assembly 280 to ensure the flushing, cleaning, and decontaminating suspension 22 is sprayed directly onto the inner wall (or exterior) of the graft or stent 24, 24′ being treated and is not entrained within the fluid flowing in the lumen. This prevents the need for the application of the flushing, cleaning, and decontaminating suspension 22 down the middle of the lumen and waiting for the flushing, cleaning, and decontaminating suspension 22 to disperse out to the inner wall of the graft or stent 24, 24′. While the various embodiments disclosed below show the Venturi-agitating tip assembly 280 with three (3) outlets, it is contemplated that as many as 16 outlets may be positioned about the tip of the Venturi-agitating tip assembly 280 or spraying the enriched flushing, cleaning, and decontaminating suspension. Further still, it is appreciated that the outlets may be covered with membranes that create a foam suspension upon dispensing. A method in accordance with the apparatus is also disclosed.

The flushing, cleaning, and decontaminating apparatus 100, in particular, the multi-channel arrangement 114, includes a dual lumen catheter 160 connecting the Venturi-agitating tip assembly 280 to the pressurized medical carbon dioxide (CO₂) 19 from the compressed supply unit 12 and the additional flushing, cleaning, and decontaminating solution 20 from a syringe 90. The syringe 90 includes a one-way valve 91 at its outlet to ensure that additional flushing, cleaning, and decontaminating solution 20 from the syringe 90 only flows out of the syringe 90. The dual lumen catheter 160 includes a first end (or distal end) 162 having the Venturi-agitating tip assembly 280 secured thereto and a second end (or proximal end) 164 to which the compressed supply unit 12 and the syringe 90 are fluidly connected for the passage of the pressurized medical carbon dioxide (CO₂) 19 and the additional flushing, cleaning, and decontaminating solution 20. As will be appreciated based upon the following disclosure, the dual lumen catheter 160 is connected to the Venturi-agitating tip assembly 280 by securing the Venturi-agitating tip assembly 280 to a first lumen 172 and a second lumen 174 of the dual lumen catheter 160, respectively. The provision of the Venturi-agitating tip assembly 280 at the very end of the dual lumen catheter 160 allows for the mixing of the pressurized medical carbon dioxide (CO₂) 19 and the additional flushing, cleaning, and decontaminating solution 20 immediately adjacent to the discharge point.

A micro-hose 156 connects the compressed supply unit 12 to the first lumen 172 of the dual lumen catheter 160 at a proximal second end 164 of the dual lumen catheter 160 for the transmission of the pressurized medical carbon dioxide (CO₂) 19 from compressed supply unit 12 to the Venturi-agitating tip assembly 180. The multi-channel arrangement 114 further includes a first stopcock 130 that is positioned between the micro-hose 156 and the first lumen 172 of the dual lumen catheter 160. The first stopcock 130 allows for three flow options; that is a first flow option for free flow between the micro-hose 156 and the first lumen 172 of the dual lumen catheter 160, a second option for the flow from the micro-hose 156 to a discharge tube 132 that is used during purging procedures, and third option blocking the flow at the end of the micro-hose 156.

As such, pressurized medical carbon dioxide (CO₂) 19 leaving the compressed supply unit 12 enters the first lumen 172 of the dual lumen catheter 160 via the micro-hose 156. After passing through the first lumen 172 of the dual lumen catheter 160, the pressurized medical carbon dioxide (CO₂) 19 enters the Venturi-agitating tip assembly 180 of the suspension delivery catheter. As will be explained below in greater detail, the flushing, cleaning, and decontaminating suspension 22 composed of the pressurized medical carbon dioxide (CO₂) 19 and the additional flushing, cleaning, and decontaminating solution 20 generated at the Venturi-agitating tip assembly 180 is directly applied to a graft or stent requiring treatment with the flushing, cleaning, and decontaminating suspension 22.

As to the connection of the additional flushing, cleaning, and decontaminating solution 20 to the suspension delivery catheter, the additional flushing, cleaning, and decontaminating solution 20 is delivered to the second lumen 174 of the dual lumen catheter 160 at the proximal second end 164 thereof, and ultimately to the Venturi-agitating tip assembly 180, via a container, in particular, the syringe 90, connected to the second lumen 174 of the dual lumen catheter 160 by a supply line 116. As with the micro-hose 156, the multi-channel arrangement 114 further includes a stopcock 134 that is positioned between the syringe 90 and the second lumen 174 of the dual lumen catheter 160. The stopcock 134 allows for three flow options; that is a first flow option for free flow between the syringe 90 and the second lumen 174 of the dual lumen catheter 160, a second option for the flow from the syringe 90 to a discharge tube 136 that is used during purging procedures, and a third option blocking the flow at the end of the syringe 90.

As mentioned above, the syringe 90 includes a one-way valve 91 at its outlet to ensure that additional flushing, cleaning, and decontaminating solution 20 from the syringe 90 only flows out of the syringe 90, preventing reflux back into the syringe 90 or the compressible syringe of the compressed supply unit 12. After passing through the second lumen 174 of the dual lumen catheter 160, the additional flushing, cleaning, and decontaminating solution 20 from the syringe 90 travels into the Venturi-agitating tip assembly 280 where it is combined with pressurized medical carbon dioxide (CO₂) 19 from the compressed supply unit 12 to form a flushing, cleaning, and decontaminating suspension 22

Considering the inclusion of the stopcocks discussed above, the operator has the option of supplying both the pressurized medical carbon dioxide (CO₂) 19 and the additional flushing, cleaning, and decontaminating solution 20 at the same time for mixing the Venturi-agitating tip assembly 280 or individual delivery by closing off one of the stopcocks 130, 134 and allowing for only flow of one of the pressurized medical carbon dioxide (CO₂) 19 and the additional flushing, cleaning, and decontaminating solution 20.

In accordance with a disclosed embodiment, one-way valves 138, 139 may be positioned along both the first lumen 172 and the second lumen 174 near to where they are respectively connected to the Venturi-agitating tip assembly 280.

The Venturi-agitating tip assembly 280 results in the spray of the flushing, cleaning, and decontaminating suspension 22 at an angle relative to a central axis of the Venturi-agitating tip assembly 280. In accordance with a preferred embodiment, and as will be discussed below with regard to the various embodiments of the Venturi-agitating tip assembly 280, the Venturi-agitating tip assembly 280 includes outlets 228 directing the flushing, cleaning, and decontaminating suspension 22 at an angle of 25 degrees to 65 degrees, preferably 45 degrees, relative to the central longitudinal axis of the Venturi-agitating tip assembly 280. By orienting the outlets at an angle as disclosed herein, the flushing, cleaning, and decontaminating suspension 22 is directed toward the walls of the graft or stent 24, 24′ into which it is being dispensed.

It is appreciated various Venturi-agitating tip assemblies and enriched flushing, cleaning, and decontaminating suspension generating structures may be employed in accordance with the present invention. These embodiments are described below with reference to the distal ends of the dual lumen catheters to which they are attached. The remainder of the apparatus not disclosed with reference to these embodiments in the same as disclosed above with reference to FIGS. 1-3. In accordance with a first embodiment as shown with reference to FIGS. 4A-4C, the Venturi-agitating tip assembly 380 employs a Venturi arrangement with a mixing chamber 324. The Venturi-agitating tip assembly 380 has a proximal first end 380a and a distal second end 380b. The Venturi-agitating tip assembly 380 includes a hollow cylindrical elongated body 310 having a proximal first end 312, which coincides with the proximal first end 380a of the Venturi-agitating tip assembly 380, and a distal second end 314. The proximal first end 380a of the Venturi-agitating tip assembly 380 includes a multi-channel arrangement 381 including first and second inputs 316, 318 for attachment to the dual lumen catheter 360. The first and second inputs 316, 318 respectively lead to a first channel 320 and a second channel 322 of the multi-channel arrangement 381 of the Venturi-agitating tip assembly 380. The first and second channels 320, 322 lead to, and are in fluid communication with, a mixing chamber 324 (which also forms part of the multi-channel arrangement 381) located in the central portion 326 of the Venturi-agitating tip assembly 380, that is, between the proximal first end 380a and the distal second end 380b. Located at the distal second end 380b of the Venturi-agitating tip assembly 380, and secured to the distal second end 314 of the elongated body 310, is a spray tip 328 directing the enriched flushing, cleaning, and decontaminating suspension in a spray pattern onto the inner lumen of a graft or stent 24, 24′ or the exterior of the graft or stent 24, 24′. As discussed above, in addition to the combination of pressurized CO₂ 19 and additional flushing, cleaning, and decontaminating solution 20 being utilized, the present system allows each of the pressurized CO₂ 19 and additional flushing, cleaning, and decontaminating solution 20 to be used individually.

The first channel 320 and the second channel 322 are interconnected in a manner creating a Venturi effect causing the pressurized medical carbon dioxide (CO₂) 19 to effectively pull the additional flushing, cleaning, and decontaminating solution 20 through the second channel 322 and into the mixing chamber 324. This is achieved by providing the first channel 320 with a reduced diameter as it extends from the proximal first end 312 of the elongated body 310 (that is, the first end 320a of the first channel 320) to the central portion 326 of the Venturi-agitating tip assembly 380 (that is, the second end 320b of the first channel 320). In accordance with a preferred embodiment, the diameter of the first channel 320 decreases from a diameter of 0.038 inches adjacent the proximal first end 312 of the elongated body 310 to a diameter of 0.017 inches adjacent the mixing chamber 324.

As mentioned above, the second channel 322 is in fluid communication with the first channel 320. This is achieved by the provision of a transverse channel 330 connecting the second end 320b of the first channel 320 with the second end 322b of the second channel 322. In particular, the second channel 322 includes a first end 322a adjacent the proximal first end 312 of the elongated body 310 and a second end 322b adjacent the mixing chamber 324 (although not directly in fluid communication with the mixing chamber 324) and the transverse channel 330. In accordance with a preferred embodiment, the diameter of the second channel 322 is 0.031 inches and remains consistent as it extends from the first end 322a thereof to the second end 322b thereof.

The first lumen 372 of the dual lumen catheter 360 supplies the pressurized medical carbon dioxide (CO₂) 19 and the second lumen 374 supplies the additional flushing, cleaning, and decontaminating solution 20. As such, the first lumen 372 is connected to, and in fluid communication with, the first channel 320 of the Venturi-agitating tip assembly 380 and the second lumen 374 is connected to, and in fluid communication with, the second channel 322 of the Venturi-agitating tip assembly 380. In practice, the additional flushing, cleaning, and decontaminating solution 20 from the syringe 90 travels through the second lumen 374 of the dual lumen catheter 360 and into the second channel 322 when pressurized medical carbon dioxide (CO₂) 19 enters the first channel 320 and passes the transverse channel 330 as it flows into the mixing chamber 324 after being actuated and released from the compressed supply unit 12. The pressurized medical carbon dioxide (CO₂) 19 entering, and passing through, the Venturi-agitating tip assembly 380 imparts negative pressure on the additional flushing, cleaning, and decontaminating solution 20 in the syringe 90 and draws the additional flushing, cleaning, and decontaminating solution 20 from the syringe 90 through the second channel 322, through the second lumen 374 of the dual lumen catheter 360, through the transverse channel 330, and into the mixing chamber 324 due to the Venturi effect. The additional flushing, cleaning, and decontaminating solution 20 and the pressurized medical carbon dioxide (CO₂) 19 are then mixed within the mixing chamber 324 to form an enriched flushing, cleaning, and decontaminating suspension. The syringe plunger is used to regulate or stop the flow of additional flushing, cleaning, and decontaminating solution 20 from the syringe 90.

The pressurized medical carbon dioxide (CO₂) 19 and additional flushing, cleaning, and decontaminating solution 20 mixing in the mixing chamber 324 are then forced through the spray tip 328 from which the enriched flushing, cleaning, and decontaminating suspension is sprayed upon the inner lumen of a graft or stent 24, 24′ or the exterior of the graft or stent 24, 24'. The spray tip 328 includes a plurality of outlets 328a oriented at an angle of 25 degrees to 65 degrees, preferably 45 degrees, relative to the central longitudinal axis of the Venturi-agitating tip assembly 380. The force of the pressurized medical carbon dioxide (CO₂) 19 traveling through the Venturi-agitating tip assembly 380 and exiting through the spray tip 328 as part of an enriched flushing, cleaning, and decontaminating suspension projects the enriched flushing, cleaning, and decontaminating suspension from the distal second end 384 of the Venturi-agitating tip assembly 380 as a spray and onto the inner lumen of a graft or stent 24, 24′ or the exterior of the graft or stent 24, 24′. As discussed above, in addition to the combination of pressurized CO₂ 19 and additional flushing, cleaning, and decontaminating solution 20 being utilized, the present system allows each of the pressurized CO₂ 19 and additional flushing, cleaning, and decontaminating solution 20 to be used individually.

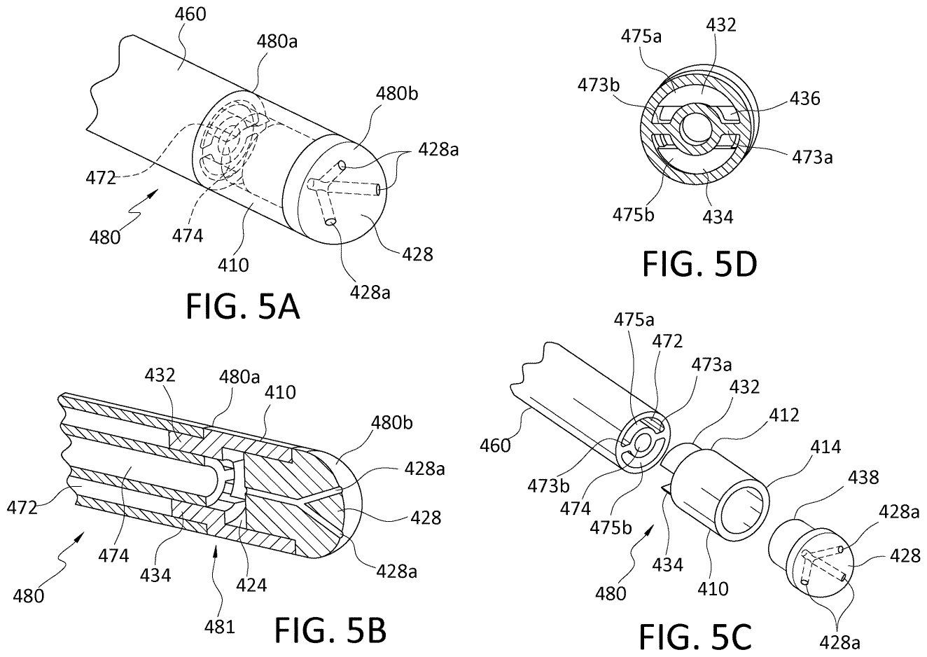

In accordance with a second embodiment as shown with reference to FIGS. 5A-5D , a Venturi-agitating tip assembly 480 employs a spray tip 428 in conjunction with a multi-channel arrangement 481 where the pressurized medical carbon dioxide (CO₂) 19 and the additional flushing, cleaning, and decontaminating solution 20 are mixed and forced through the spray tip 428. The Venturi-agitating tip assembly 480 includes a proximal first end 480a and a distal second end 480b. The Venturi-agitating tip assembly 480 includes a hollow cylindrical elongated body 410 having a proximal first end 412, which coincides with the proximal first end 480a of the Venturi-agitating tip assembly 480, and a distal second end 414. The Venturi-agitating tip assembly 480 is adapted for use with a dual lumen catheter 460, in particular a dual lumen catheter having concentric lumens, wherein the outer first lumen 472 is annular shaped for the passage of pressurized medical carbon dioxide (CO₂) 19 (and has an outer diameter of 0.092 inches at the outer wall thereof and an inner diameter of 0.042 inches at the inner wall thereof) and the inner second lumen 474 is circular shaped for the passage of the additional flushing, cleaning, and decontaminating solution 20 (and has a diameter of 0.030 inches). The inner second lumen 474 is supported within the outer first lumen 472 by first and second radially extending rib members 473a, 473b (each having a thickness of 0.006 inches) that extend from the outer surface of the second lumen 474 to the inner surface of the outer first lumen 472. In this way the outer first lumen 472 is divided into first and second semicircular passageways 475a, 475b.

The proximal first end 480a of the Venturi-agitating tip assembly 480, in particular, the proximal first end 412 of the elongated body 410 is formed with two projections 432, 434 shaped and dimensioned for engagement within the outer first lumen 472 of the catheter 460 in a manner blocking a substantial portion of the outer first lumen 472. The two projections 432, 434 are arcuate members shaped and dimensioned to respectively block substantial portions of the first and second semicircular passageways 475a, 475b while creating four small passageways 436, each of approximately 0.031 inches (along the Y-axis as shown in FIG. 5D) by 0.050 inches (along the X-axis as shown in FIG. 5D) for the passage of pressurized medical carbon dioxide (CO₂) 19 therethrough. The four small passageways 436 are defined by spaces existing between the edges of the arcuate members (that is, the two projections) 432, 434 and the first and second radially extending rib members 473a, 473b.

The remainder of the Venturi-agitating tip assembly 480 includes a central mixing chamber 424 that is in fluid communication with the second lumen 474 and the four small passageways 436 feeding pressurized medical carbon dioxide (CO₂) 19 from the first lumen 472. Secured to, and closing off, the second end 414 of the elongated body 410 is a spray tip 428, which is thereby at the distal second end 480b of the Venturi-agitating tip assembly 480. Attachment of the spray tip 428 to the elongated body 410 is achieved by providing the spray tip 428 with a projection 438 that seats within the opening at the second end 414 of the elongated body 410.

The first lumen 472 and the second lumen 474 are interconnected in a manner causing the pressurized medical carbon dioxide (CO₂) 19 to effectively pull the additional flushing, cleaning, and decontaminating solution 20 through the second lumen 474 and into the mixing chamber 424. In practice, the additional flushing, cleaning, and decontaminating solution 20 from the syringe 90 travels through the second lumen 474 of the dual lumen catheter 460 and into the mixing chamber 424 when pressurized medical carbon dioxide (CO₂) 19 passes through the four small passageways 436 and enters the mixing chamber 424 (where the additional flushing, cleaning, and decontaminating solution 20 from the syringe 90 and the pressurized medical carbon dioxide (CO₂) 19 mix to form an enriched flushing, cleaning, and decontaminating suspension) after being actuated and released from compressed supply unit 12. The pressurized medical carbon dioxide (CO₂) 19 entering, and passing through, the mixing chamber 424 imparts negative pressure on the additional flushing, cleaning, and decontaminating solution 20 in syringe 90 and draws the additional flushing, cleaning, and decontaminating solution 20 from the syringe 90 through the second lumen 474 and into the mixing chamber 424. The syringe plunger is used to regulate or stop the flow of the additional flushing, cleaning, and decontaminating solution 20 from the syringe 90.

The pressurized medical carbon dioxide (CO₂) 19 and additional flushing, cleaning, and decontaminating solution 20 mixing in the mixing chamber 424 are then forced through the spray tip 428 from which an enriched flushing, cleaning, and decontaminating suspension is sprayed upon the inner surface of a lumen of the graft or stent 24, 24′ or the exterior of the graft or stent 24, 24′. The spray tip 428 includes a plurality of outlets 428a oriented at an angle of 25 degrees to 65 degrees, preferably 45 degrees, relative to the central longitudinal axis of the Venturi-agitating tip assembly 480. The force of the pressurized medical carbon dioxide (CO₂) 19 traveling through the Venturi-agitating tip assembly 480 and exiting through the spray tip 428 as part of an enriched flushing, cleaning, and decontaminating suspension projects the enriched flushing, cleaning, and decontaminating suspension from the distal second end 484 of the Venturi-agitating tip assembly 480 as a spray and onto the inner lumen of a graft or stent 24, 24′ or the exterior of the graft or stent 24, 24′. As discussed above, in addition to the combination of pressurized CO₂ 19 and additional flushing, cleaning, and decontaminating solution 20 being utilized, the present system allows each of the pressurized CO₂ 19 and additional flushing, cleaning, and decontaminating solution 20 to be used individually.

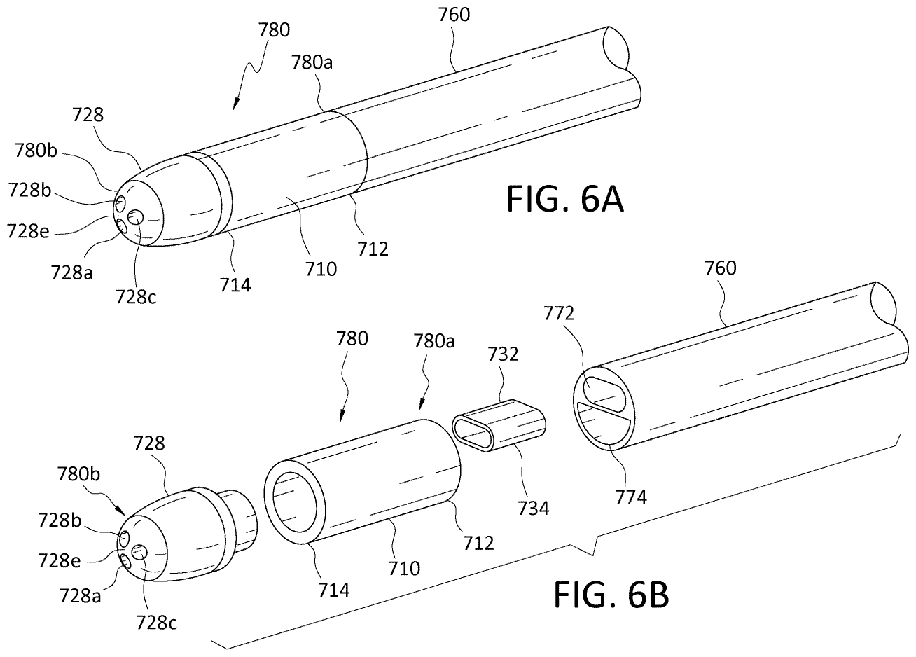

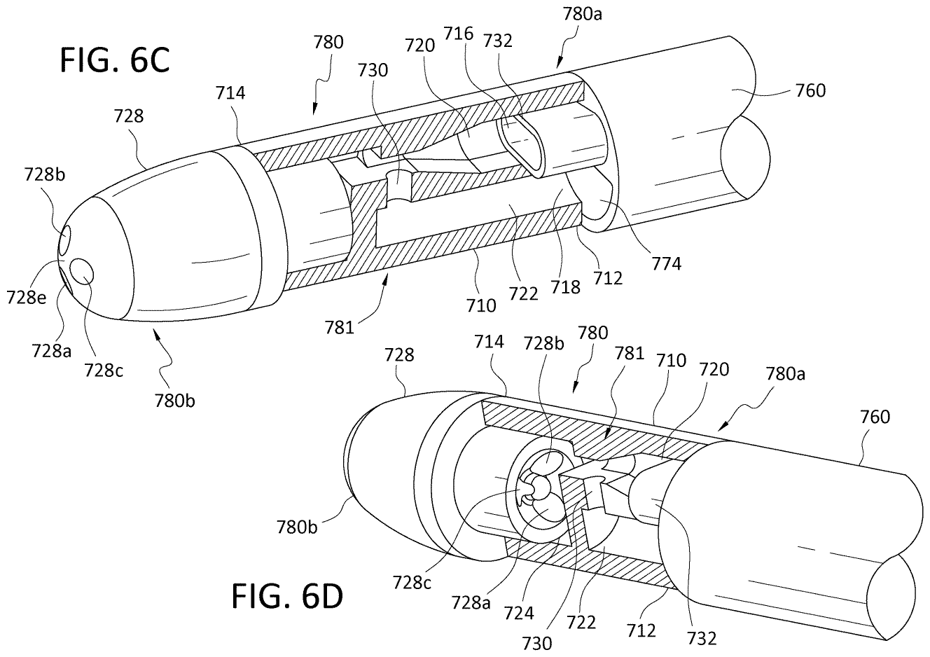

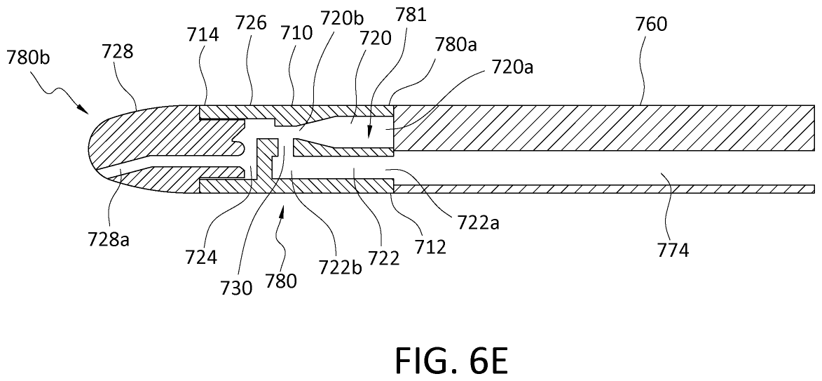

In accordance with a third embodiment as shown with reference to FIGS. 6A-6E, a Venturi-agitating tip assembly 780 employs a tip 728 in conjunction with a multi-channel arrangement 781 where the pressurized medical carbon dioxide (CO₂) 19 and additional flushing, cleaning, and decontaminating solution 20 are mixed to form an enriched flushing, cleaning, and decontaminating suspension and forced through the tip 728. The Venturi-agitating tip assembly 780 includes a proximal first end 780a and a distal second end 780b. The Venturi-agitating tip assembly 780 includes a hollow cylindrical elongated body 710 having a proximal first end 712, which coincides with the proximal first end 780a of the Venturi-agitating tip assembly 780, and a distal second end 714. The Venturi-agitating tip assembly 780 is adapted for use with a multi-lumen catheter 760, in particular a dual lumen catheter having parallel lumens, wherein the first lumen 772 is oval shaped and is dimensioned for the passage of pressurized medical carbon dioxide (CO₂) 19 and the second lumen 774 is semi-circular shaped and is dimensioned for the passage of the additional flushing, cleaning, and decontaminating solution 20.

The proximal first end 712 of the elongated body 710 at the proximal first end 780a of the Venturi-agitating tip assembly 780 includes first and second inputs 716, 718 for attachment to the multi-lumen catheter 760. The first input 716 leads to a first channel 720 and the second input 718 to a second channel 722. The proximal first end 712 of the elongated body 710 at the proximal first end 780a of the Venturi-agitating tip assembly 780 is formed with an oval tubular projection 732, defining the first input 716. The oval tubular projection 732 is shaped and dimensioned for engagement within the first lumen 772 of the catheter 760 in a manner allowing for the flow of fluid from the first lumen 772 and into the Venturi-agitating tip assembly 780. The oval tubular projection 732 is shaped and dimensioned to fit within the first lumen 772 while maintaining a passageway for the passage of pressurized medical carbon dioxide (CO₂) 19 therethrough.

The first and second channels 720, 722 lead to, and are in fluid communication with, a mixing chamber 724 located in the central portion 726 of the Venturi-agitating tip assembly 780, that is, between the proximal first end 712 and the distal second end 714 of the elongated body. Secured to the distal second end 714 of the elongated body 710, and positioned at the distal second end 780b of the Venturi-agitating tip assembly, is a tip 728 having three outlets 728a, 728b, 728c extending from the mixing chamber 724 to the exterior at the distal end of the Venturi-agitating tip assembly 780 at an angle of 25 degrees to 65 degrees, preferably 45 degrees, relative to the central longitudinal axis of the Venturi-agitating tip assembly 780.

The first channel 720 and the second channel 722 are interconnected in a manner creating a Venturi effect causing the pressurized medical carbon dioxide (CO₂) 19 to effectively pull the additional flushing, cleaning, and decontaminating solution 20 through the second channel 722 and into the mixing chamber 724. This is achieved by providing the first channel 720 with a reduced diameter (decreasing from 0.038 inches to 0.017 inches) as it extends from the proximal first end 712 of the elongated body 710 (that is, the first end 720a of the first channel 720) to the central portion 726 of the Venturi-agitating tip assembly 780 (that is, the second end 720b of the first channel 720). In accordance with a preferred embodiment, the diameter of the first channel 720 decreases from a diameter of 0.038 inches adjacent the proximal first end 712 of the elongated body 710 to a diameter of 0.017 inches adjacent the mixing chamber 724.

As mentioned above, the second channel 722 is in fluid communication with the first channel 720. This is achieved by the provision of a transverse channel 730 connecting the second end 720b of the first channel 720 with the second end 722b of the second channel 722. In particular, the second channel 722 includes a first end 722a adjacent the proximal first end 712 of the elongated body 710 and a second end 722b adjacent the mixing chamber 724 (although not directly in fluid communication with the mixing chamber 724) and the transverse channel 730. In accordance with a preferred embodiment, the diameter of the second channel 722 is 0.047 inches and remains consistent as it extends from the first end 722a thereof to the second end 722b thereof.

The first lumen 772 supplies the pressurized medical carbon dioxide (CO₂) 19 and the second lumen 774 supplies the additional flushing, cleaning, and decontaminating solution 20. As such, the first lumen 772 is connected to, and in fluid communication with, the first channel 720 of the Venturi-agitating tip assembly 780. The second lumen 774 is connected to, and in fluid communication with, the second channel 722 of the Venturi-agitating tip assembly 780. In practice, the additional flushing, cleaning, and decontaminating solution 20 from syringe 90 travels through second lumen 774 of multi-lumen catheter 760 and into the second channel 722 when pressurized medical carbon dioxide (CO₂) 19 enters the first channel 720 and passes the transverse channel 730 (having a size of 0.020 inches) into the mixing chamber 724 after being actuated and released from compressed supply unit 12. The pressurized medical carbon dioxide (CO₂) 19 entering, and passing through, the Venturi-agitating tip assembly 780 imparts negative pressure on the additional flushing, cleaning, and decontaminating solution 20 in syringe 90 and draws the additional flushing, cleaning, and decontaminating solution 20 from the syringe 90 through second channel 722, through the second lumen 774 of the dual lumen catheter 760, and into the mixing chamber 724 due to the Venturi effect. The syringe plunger is used to regulate or stop the flow of additional flushing, cleaning, and decontaminating solution 20 from the syringe 90.

The pressurized medical carbon dioxide (CO₂) 19 and additional flushing, cleaning, and decontaminating solution 20 mixing in the mixing chamber 724 form an enriched flushing, cleaning, and decontaminating suspension that is then forced through the outlets 728a-c of the spray tip 728. The force of the suspension traveling through the Venturi-agitating tip assembly 780 and exiting through the spray tip 728 as part of an enriched flushing, cleaning, and decontaminating suspension projects the enriched flushing, cleaning, and decontaminating suspension from the distal second end 784 of the Venturi-agitating tip assembly 780 as a spray and onto the inner lumen of a graft or stent 24, 24′ or the exterior of the graft or stent 24, 24′.

While the outlets of the spray tip in the embodiments disclosed above with reference to FIGS. 4A-C, FIGS. 5A-D, and 6A-E are positioned along a forward portion of the spray tip, it is appreciated that the outlets of the spray tip could be positioned along the outer circumferential wall of the spray tip as shown in FIG. 3. Through the utilization of either position for the outlets, the enriched flushing, cleaning, and decontaminating suspension is sprayed from the Venturi-agitating tip assembly onto the inner lumen of a graft or stent 24, 24′ or the exterior of the graft or stent 24, 24′. As discussed above, in addition to the combination of pressurized CO₂ 19 and additional flushing, cleaning, and decontaminating solution 20 being utilized, the present system allows each of the pressurized CO₂ 19 and additional flushing, cleaning, and decontaminating solution 20 to be used individually.

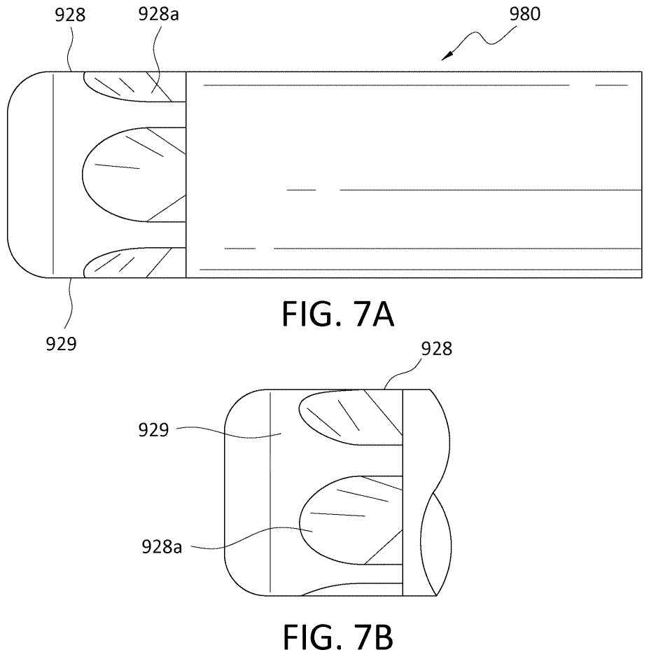

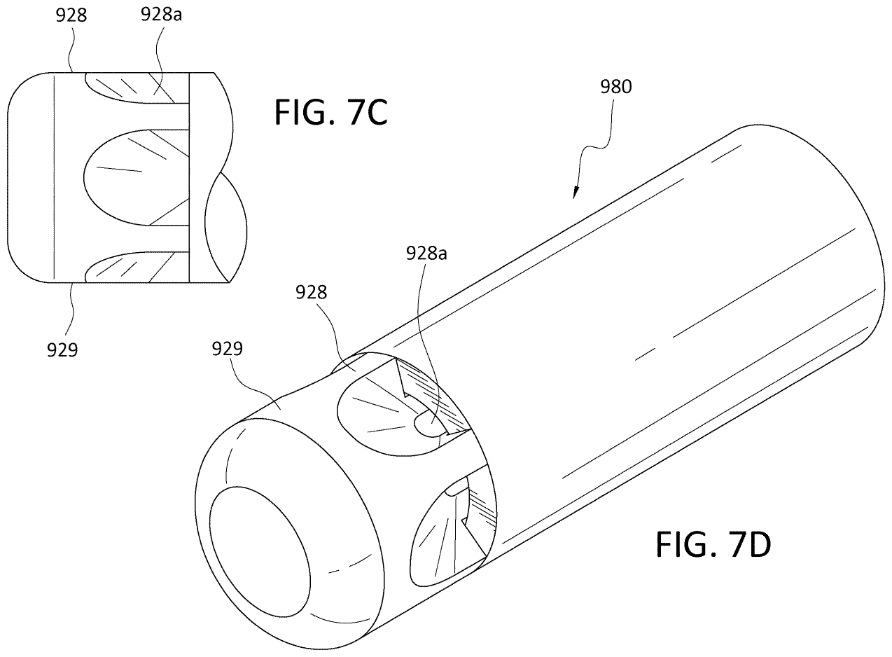

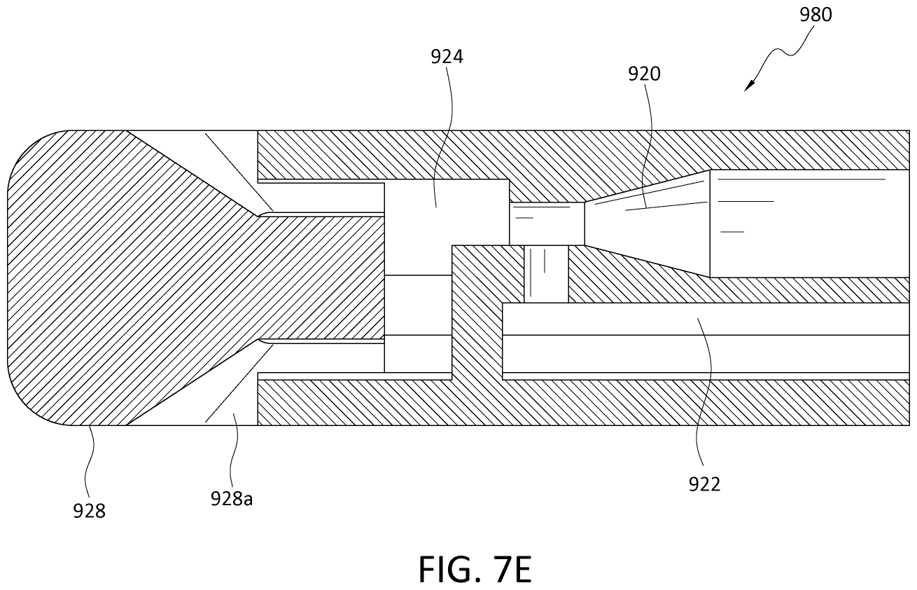

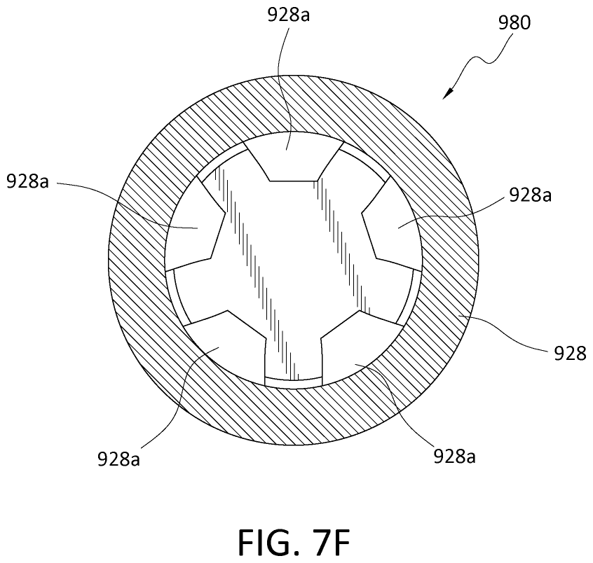

For example, and with reference to FIG. 7A-7F, a fourth embodiment with outlets 928a of the spray tip 928 positioned along the outer circumferential wall 929 of the spray tip 928. Those portions of the Venturi-agitating tip assembly 980 proximal to the spray tip 928, for example, the mixing chamber 924, the first channel 920, and the second channel 922 are the same as found in the embodiment disclosed with reference to FIGS. 4A-4C. However, the spray tip 928 includes a plurality of circumferentially oriented outlets 928a positioned so as to dispense the enriched flushing, cleaning, and decontaminating suspension through the outer circumferential wall 929 of the spray tip 928.

In accordance with the various embodiments described above, the flushing, cleaning, and decontaminating suspension 22 exiting the Venturi-agitating tip assembly is directed to a graft or stent 24, 24′ requiring treatment. In accordance with a preferred embodiment, the method for treatment in accordance with the present invention is achieved in the following manner. The first end of the suspension delivery catheter, that is, the Venturi-agitating tip assembly is introduced into a graft or stent 24, 24′ requiring treatment such that the first end of the Venturi-agitating tip assembly is positioned beyond the section of graft or stent 24, 24′ requiring treatment. The second end of the delivery catheter is coupled to the compressed supply unit 12 and the syringe. At this point, the compressed supply unit 12 is actuated to supply pressurized medical carbon dioxide (CO₂) 19 to the suspension delivery catheter and an enriched flushing, cleaning, and decontaminating suspension is produced at the Venturi-agitating tip assembly of the suspension delivery catheter. The enriched flushing, cleaning, and decontaminating suspension sprays from the first end of the Venturi-agitating tip assembly into the section of graft or stent 24, 24′ requiring treatment.

While this detailed description has set forth particularly preferred embodiments of the apparatus of this invention, numerous modifications and variations of the structure of this invention, all within the scope of the invention, will readily occur to those skilled in the art. Accordingly, it is understood that this description is illustrative only of the principles of the invention and is not limitative thereof.

Although specific features of the invention are shown in some of the drawings and not others, this is for convenience only, as each feature may be combined with any and all of the other features in accordance with this invention.

While the preferred embodiments have been shown and described, it will be understood that there is no intent to limit the invention by such disclosure, but rather, it is intended to cover all modifications and alternative constructions falling within the spirit and scope of the invention. Please add text for any optional specification sub-header in this area.

1. A flushing, cleaning, and decontaminating apparatus for flushing, cleaning, and decontaminating grafts and stents, comprising:
a multi-channel arrangement at a proximal first end thereof and a Venturi-agitating tip assembly at a tip at a distal second end thereof;
a compressed supply unit fluidly connected to the multi-channel arrangement for supplying pressurized medical carbon dioxide (CO₂) to the Venturi-agitating tip assembly and; and
an additional flushing, cleaning, and decontaminating solution fluidly connected to the multi-channel arrangement for supplying additional flushing, cleaning, and decontaminating solution to the Venturi-agitating tip assembly;
wherein pressurized medical carbon dioxide (CO₂) from the compressed supply unit and the additional flushing, cleaning, and decontaminating solution are combined within the Venturi-agitating tip assembly in a manner generating a cleaning medium that is ultimately dispensed from therefrom.

2. The flushing, cleaning, and decontaminating apparatus according to claim 1, wherein the flushing, cleaning, and decontaminating apparatus allows each of the pressurized CO₂ and additional flushing, cleaning, and decontaminating solution to be used individually.

3. The flushing, cleaning, and decontaminating apparatus according to claim 1, wherein the compressed supply unit includes an inlet port to which a pressurized gas cylinder containing medical carbon dioxide (CO₂) is selectively connected, and an outlet port in communication with the inlet port, and ultimately the pressurized gas cylinder containing medical carbon dioxide (CO₂).

4. The flushing, cleaning, and decontaminating apparatus according to claim 3, wherein the outlet port is provided with a Luer connection for secure and selective attachment of an outlet tube, and the outlet tube is secured to the multi-channel arrangement for the supply of pressurized medical carbon dioxide (CO₂) to thereto.

5. The flushing, cleaning, and decontaminating apparatus according to claim 1, wherein outlets at a tip of the Venturi-agitating tip assembly are oriented such that a flushing, cleaning, and decontaminating suspension is sprayed at an angle of 25 degrees to 65 degrees relative to a longitudinal axis of the Venturi-agitating tip assembly to ensure the flushing, cleaning, and decontaminating suspension is sprayed directly onto an inner wall of the graft or stent being treated and is not entrained within fluid flowing.

6. The flushing, cleaning, and decontaminating apparatus according to claim 1, wherein the multi-channel arrangement includes a dual lumen catheter that connects the Venturi-agitating tip assembly to the pressurized medical carbon dioxide (CO₂) from the compressed supply unit and the additional flushing, cleaning, and decontaminating solution from a syringe.

7. The flushing, cleaning, and decontaminating apparatus according to claim 6, wherein the syringe includes a one-way valve at its outlet to ensure that additional flushing, cleaning, and decontaminating solution from the syringe only flows out of the syringe.

8. The flushing, cleaning, and decontaminating apparatus according to claim 6, wherein a first stopcock is positioned between a micro-hose and a first lumen of the dual lumen catheter.

9. The flushing, cleaning, and decontaminating apparatus according to claim 8, wherein the first stopcock allows for three flow options comprising a first flow option for free flow between the micro-hose and the first lumen of the dual lumen catheter, a second option for the flow from the micro-hose to a discharge tube that is used during purging procedures, and third option blocking the flow at the end of the micro-hose.

10. The flushing, cleaning, and decontaminating apparatus according to claim 8, wherein the additional flushing, cleaning, and decontaminating solution is delivered to a second lumen of the dual lumen catheter at the proximal first end thereof, and ultimately to the Venturi-agitating tip assembly, via the syringe, connected to the second lumen of the dual lumen catheter by a supply line.

11. The flushing, cleaning, and decontaminating apparatus according to claim 10, wherein a second stopcock is positioned between the syringe and the second lumen of the dual lumen catheter.

12. The flushing, cleaning, and decontaminating apparatus according to claim 11, wherein the second stopcock allows for three flow options comprising a first flow option for free flow between the syringe and the second lumen of the dual lumen catheter, a second option for the flow from the syringe to a discharge tube that is used during purging procedures, and third option blocking the flow at the end of the syringe.

13. The flushing, cleaning, and decontaminating apparatus according to claim 6, wherein the additional flushing, cleaning, and decontaminating solution is delivered to a second lumen of the dual lumen catheter at the proximal first end thereof, and ultimately to the Venturi-agitating tip assembly, via the syringe, connected to the second lumen of the dual lumen catheter by a supply line.

14. The flushing, cleaning, and decontaminating apparatus according to claim 13, wherein a second stopcock is positioned between the syringe and the second lumen of the dual lumen catheter.

15. The flushing, cleaning, and decontaminating apparatus according to claim 14, wherein the second stopcock allows for three flow options comprising a first flow option for free flow between the syringe and the second lumen of the dual lumen catheter, a second option for the flow from the syringe to a discharge tube that is used during purging procedures, and third option blocking the flow at the end of the syringe.

16. The flushing, cleaning, and decontaminating apparatus according to claim 1, wherein the proximal first end of the Venturi-agitating tip assembly includes first and second inputs for attachment to a dual lumen catheter.

17. The flushing, cleaning, and decontaminating apparatus according to claim 16, wherein the first and second inputs respectively lead to a first channel and a second channel of the Venturi-agitating tip assembly, and the first and second channels lead to, and are in fluid communication with, a mixing chamber located in a central portion of the Venturi-agitating tip assembly between the proximal first end and the distal second end.
